(19) United States
(12) Plant Patent Application Publication
(10) Pub. No.: US 2002/0148024 P1
(43) Pub. Date: Oct. 10, 2002

(54) Kalanchoe plant named Ridge

(76) Inventor: Andre Smaal, Aalsmeer (NL)

Correspondence Address: ROCKEY, MILNAMOW & KATZ, LTD., Two Prudential Plaza, Suite 4700, 180 North Stetson Avenue, Chicago, IL, 60601, US

(21) Appl. No.: 09/782,641

(22) Filed: Feb. 13, 2001

(51) Int. Cl. A01H 5/00

(52) U.S. Cl. PLT/338

(57) ABSTRACT

A new and distinct cultivar of Kalanchoe named Ridge is provided. This new cultivar is characterized by its bright yellow flowers, dark green foliage, compact growth habit and excellent basal branching.

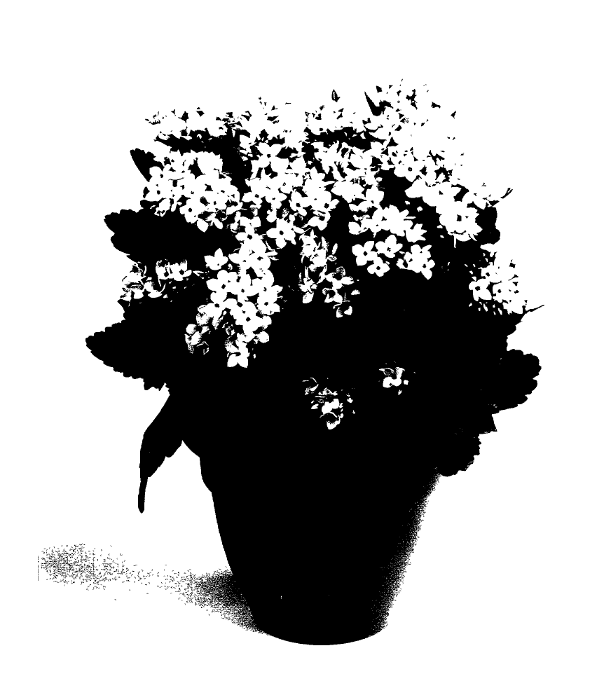

### BACKGROUND OF THE INVENTION

The present invention comprises a new and distinct Kalanchoe plant, botanically known as Kalanchoe blossfeldiana, and hereinafter referred to by the cultivar name Ridge.

The new cultivar was developed by the inventor through a controlled breeding program during August 1996 in The Netherlands. The female (seed) parent of Ridge was the proprietary Kalanchoe cultivar designated 95114-2, characterized by its yellow flowers and dark green foliage. The male (pollen) parent of Ridge was the proprietary Kalanchoe cultivar designated 94036, characterized by its yellow flowers, dark green foliage and good branching habit. Ridge was discovered and selected as a flowering plant within the progeny of the stated cross by the inventor in June 1997.

Asexual reproduction of the new cultivar by terminal cuttings has demonstrated that the characteristics of the new cultivar as herein described are firmly fixed and retained through successive generations of such asexual propagation.

### SUMMARY OF THE INVENTION

It was found that the cultivar of the present invention:

(a) exhibits bright yellow flowers,

(b) forms dark green foliage,

(c) exhibits a good basal branching character and

(d) exhibits a compact upright growth habit.

The new cultivar of the present invention can be compared to Goldstrike (U.S. Plant Pat. No. 6,632). In side by side comparisons with Goldstrike, it is found that the new cultivar is more compact, has fewer branches and exhibits flowers of a lighter yellow color. A detailed comparison can be found in Table A.

### BRIEF DESCRIPTION OF PHOTOGRAPH

The accompanying photograph shows as nearly true as it is reasonably possible to make the same in color illustrations of this type, typical flower and foliage characteristics of the new cultivar. The plants were grown in a greenhouse at West Chicago, Ill., U.S.A.

### DETAILED DESCRIPTION

The Ridge cultivar has not been observed under all possible environmental conditions to date. Accordingly, it is possible that the phenotype may vary somewhat with variations in the environment, such as temperature, light intensity, and day length.

The chart used in the identification of colors described herein is the R.H.S. Colour Chart of The Royal Horticultural Society, London, England. The color values were determined on Jan. 4, 2001 between 9:00 and 9:30 am under natural daylight. The plants used for the following observations and measurements were produced from cuttings taken from stock plants and grown in West Chicago, Ill. in 10 cm pots for 16 weeks. Plants were grown in a double poly carbonate covered greenhouse under conditions comparable to those used in commercial practice while utilizing a soilless growth medium and maintaining temperatures of approximately 82° F. during the day and approximately 66° F. during the night. Plants were exposed to three weeks of long days followed by 9 weeks of short days. Growth regulators were applied as follows: B nine, 3000 ppm at days 17 and 28 and 1500 ppm day 42.

Classification:

Botanical.—Kalanchoe blossfeldiana, cultivar Ridge.

Parentage: Proprietary seedling 95114-2 (seed)×Proprietary seedling 94036 (pollen).

Propagation:

Type cutting.—Terminal tip.

Time to initiate roots.—Approximately 7-14 days with the shorter times generally being experienced in the summer and the longer times in the winter.

Time to develop roots.—Approximately 21 days.

Root description.—Fibrous, branching.

Plant Description:

Habit of growth.—Moderately vigorous with good basal branching. A mature plant, at 16 weeks after the planting of an unrooted cutting, commonly measures approximately 18.7 cm in height and approximately 15.3 cm in width.

Branching habit.—Typically forms one main stem 13 cm in length and 1 cm in diameter and 3 to 5 lateral branches approximately 11 cm in length and 7 mm in diameter.

Plant form.—Upright and mounded.

Foliage.—Simple, opposite. Shape: Ovate, rounded apex, rounded base, crenate margin.

Texture.—Glabrous, succulent. Size of mature foliage: Approximately 9 cm in length and approximately 7 cm in width. Color of mature foliage: Upper surface closest to 139A; lower surface closest to 137B. Petiole length: Approximately 2 cm, color is 144A.

Flower Description:

Flowering habit.—Freely flowering.

Natural flowering season.—Early January. Flowering time is dependent on temperature, light intensity and other growing conditions.

Inflorescence type.—Dichasial cymes.

Flowers borne.—Above foliage.

Flower longevity.—Approximately 7 days.

Fragrance.—None.

Quantity of flowers.—Approximately 87 flowers on main stem and 29 flowers on each lateral branch.

Flower buds.—Shape: Elliptical, becoming elongated as it matures. Size just before opening:

Length is approximately 1.5 cm, width is approximately 3 mm. Color: 1 3D.

Flower.—Slaverform corolla. Approximately 1.6 cm in diameter, 3 mm in depth. Corolla tube is 9 mm long.

Petals.—Number: Four. Shape: Ovate with cuspidate apex, rounded base. Approximate size: 7 mm in length, 6 mm in width. Aspect: Slightly convex. Texture: Glabrous and somewhat iridescent.

Sepals.—Number: 4. Shape: Lanceolate with acuminate apex, entire margin. Size: 7 mm in length. Texture: Smooth. Color: Upper and lower surface: 144B.

Mature flower color.—Upper surface of petals is closest to 13B fading to 21 B at senescence; lower surface of petals is closest to 13D.

Peduncle.—Length: Approximately 2 cm in length and 4 mm in diameter.

Pedicel length.—Approximately 5 mm.

Reproductive organs.—Androecium: 8 stamens arranged in two groups of four—one group being shorter than the other. Anther shape is oblong and flat, length is approximately 0.5 mm. Pollen color is 6C. Gynoecium: Stigma shape is flat, appearance is crystalline, colorless and transparent. Style length is approximately 2 mm, color is 151C. Ovary is 4 celled, 7mm in length. Color is 144A.

Seed development: Seed production has not been observed.

Disease resistance: Resistance to pathogens has not been observed.

I claim:

1. A new and distinct cultivar of Kalanchoe named Ridge substantially as herein shown and described, which:
(a) exhibits bright yellow flowers,
(b) forms dark green foliage,
(c) exhibits a good basal branching character and
(d) exhibits a compact upright growth habit.
TABLE A

| CHARACTERISTIC | RIDGE | GOLDSTRIKE |
|---|---|---|
| PLANT HEIGHT | 18.7 CM | 23 CM |
| NUMBER OF BRANCHES | 3 | 6 |
| FLOWER DIAMETER | 7.2 CM | 6.8 CM |
| PETAL COLOR-FULLY OPENED- | 13 B | 15 A |
| UPPER SURFACE | | |
| PETAL COLOR-FULLY OPENED- | 13 D | 16 C |
| LOWER SURFACE | | |
| PETAL COLOR FADING TO | 21 B | 16 B |
